(19) United States
(12) Patent Application Publication
(10) Pub. No.: US 2018/0271506 A1
(43) Pub. Date: Sep. 27, 2018

(54) DEVICES AND METHODS FOR USE IN PERFORMING ARTHROSCOPIC TOTAL SHOULDER REPLACEMENT

(71) Applicant: Timothy G. Reish, Briarcliff Manor, NY (US)

(72) Inventor: Timothy G. Reish, Briarcliff Manor, NY (US)

(21) Appl. No.: 15/723,056

(22) Filed: Oct. 2, 2017

Related U.S. Application Data
(60) Provisional application No. 62/402,791, filed on Sep. 30, 2016.

(51) Int. Cl. A61B 17/02; A61B 1/07; A61B 1/00; A61B 1/32; A61B 1/045; A61B 1/317

(52) U.S. Cl. CPC A61B 17/025; A61B 1/07; A61B 1/00179; A61B 1/32; A61B 2017/00407; A61B 1/0016; A61B 1/045; A61B 1/317; A61B 1/00147

(57) ABSTRACT

Devices and instruments for performing complete shoulder replacement using arthroscopic techniques. The devices may include (i) an arthroscopic camera cannula inclusive of expandable flanges, (ii) arthroscopic camera support stand with a longitudinal adjustment mechanism to adjust an arthroscopic camera inward and outward within a joint, such as a shoulder joint to adjust field-of-view within the joint, (iii) arthroscopic instrument configured to apply a higher vacuum pressure, and (iv) arthroscopic working cannula with spreader members that, when spread from a closed position to an open position, form an expanded triangular area between the subscapularis and supraspinatus tendons.

### RELATED APPLICATIONS

This application claims priority to co-pending U.S. Provisional Application having Ser. No. 62/402,791 filed on Sep. 30, 2016, the contents of which are hereby incorporated by reference in their entirety.

### BACKGROUND

Shoulder replacement surgery has been performed for many years. However, very few advancements have materialized over the years. In general, shoulder surgery is currently performed by opening up the skin to expose the shoulder and cutting or detaching from the front rotator cuff tendon, called the subscapularis, by either osteotomy, tenotomy, or peeling the tendon off the bone. After the surgery, the subscapularis is repaired or reattached. As a result of cutting or detaching the subscapular, patients have to protect motion, including forced internal rotation and passive external rotation, of the shoulder for six to eight weeks. For such surgeries, patients are hospitalize anywhere between 24 and 72 hours. The cost for such surgeries is high due to the hospitalization and ability for the patient to function at normal capacity is limited during a post-surgery period due to pain medications and limited motion of the shoulder. Moreover, post-surgery rehabilitation can be timely and costly, as a patient may have to re-strengthen his or her arm due to the limited motion for the six to eight weeks.

### SUMMARY

Arthroscopic shoulder surgery for total shoulder replacement may be revolutionary in reducing down-time, cost, and hospital stay time for patients, reducing cost for insurance companies, and increasing patient processing and reducing cost for hospitals. Arthroscopic shoulder surgery is minimally invasive, so patients would be able to receive a complete shoulder replacement in same day surgery. Moreover, the ability to perform arthroscopic shoulder surgery would eliminate the need to cut or detach the subscapularis shoulder tendon when replacing a patient's shoulder, thereby providing for reduced costs and accelerated post-surgical rehabilitation for the patient. Post-surgical rehabilitation would be accelerated, for example, as rehabilitation may be started at a much more advanced level, starting with 20 pound weight curl exercises within a few days, being able to start with higher degrees of arm motion over an extended period of time sooner, and so forth. Still yet, the patient would have virtually no rotation limitations, so full range of motion for the patient may exist more rapidly, thereby reducing or eliminating loss of work time for the patient.

Arthroscopic shoulder surgery may be performed by advancing a number of devices and processes when performing the surgery. The device advancement may include (i) an addition of an arthroscope stop added to a cannula of an arthroscope instrument, (ii) an orthopedic arthroscopic surgical arm that may be used to hold and direct or aim an arthroscopic camera during surgery, thereby enabling the surgeon to used both hands during the shoulder surgery, (iii) increased suction via the arthroscopic camera device; and (iv) triangular surgical instrument with feet to expand a triangular working space opening between the subscapularis (front rotator cuff tendon) and supraspinatus (superior rotator cuff tendon). As described herein, the devices and processes provide for arthroscopic shoulder surgery to enable full shoulder replacement surgery to be performed utilizing existing shoulder replacement components, including a Glenoid component and humeral head component.

An embodiment of an arthroscopic camera cannula may include a hollow, elongated member having a first end and a second end, where the elongated member has a diameter less then approximately 4 mm to enable an arthroscopic camera optical tube to be inserted therethrough. Multiple expandable flanges may be coupled to the elongated member toward the second end. A mechanism may be in mechanical communication with the expandable flanges that, when moved, causes the expandable flanges to move between a closed position and an open position.

An embodiment of an arthroscopic camera support stand may include an articulating bar including multiple joints between a base end and a retention end. A retention member may be attached to the retention end of the articulating bar, and be configured to retain an arthroscopic camera. The retention member may include a first mechanism configured to retain the arthroscopic camera, and a second mechanism configured to longitudinally adjust position of the arthroscopic camera. In an embodiment, a rotational member may be attached to the retention member, and be configured to rotate the arthroscopic camera.

An embodiment of an arthroscopic instrument may include a housing having an inlet port and an outlet port. A first tube may be connected to the inlet port for inserting fluid into the inlet port to be fluidly transported into a joint of a patient. A second tube may be connected to the outlet port, and be configured to apply a vacuum pressure to the outlet port. A vacuum may be applied to the second tube to apply a vacuum pressure below a vacuum pressure available in an operating room.

Another embodiment of an arthroscopic working cannula may include a cannula member, a first spreader member may be attached to and extend radially along the cannula member, where the first spreader member has a first end and a second end. The second end of the first spreader member may have a curved shape to hook onto the subscapularis tendon. A second spreader member may be attached to and extend radially along the cannula member, where the second spreader member may have a first end and a second end. The second end of the second spreader member may have a curved shape to hook onto the supraspinatus tendon. The first and second spreader members may be disposed in opposing position relative to one another, and, when spread from a closed position to an open position, form an expanded triangular area between the subscapularis and supraspinatus tendons.

### BRIEF DESCRIPTION

A more complete understanding of the method and apparatus of the present invention may be obtained by reference to the following Detailed Description when taken in conjunction with the accompanying Drawings wherein:

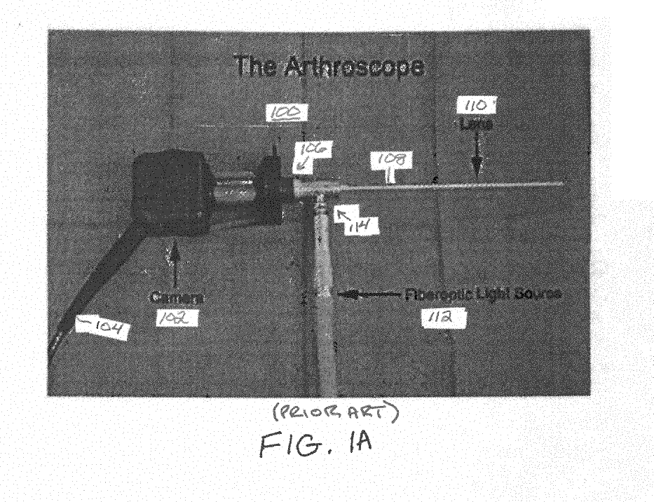

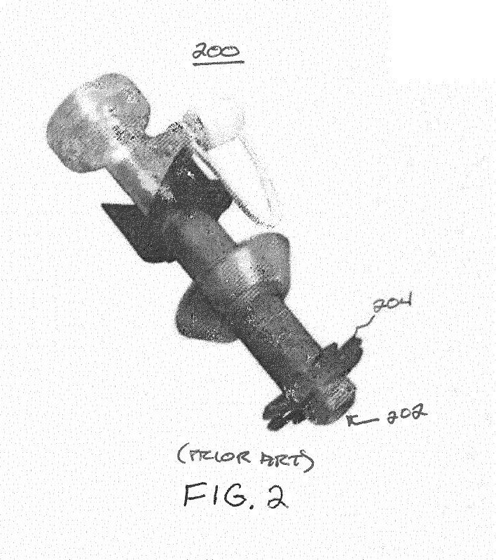

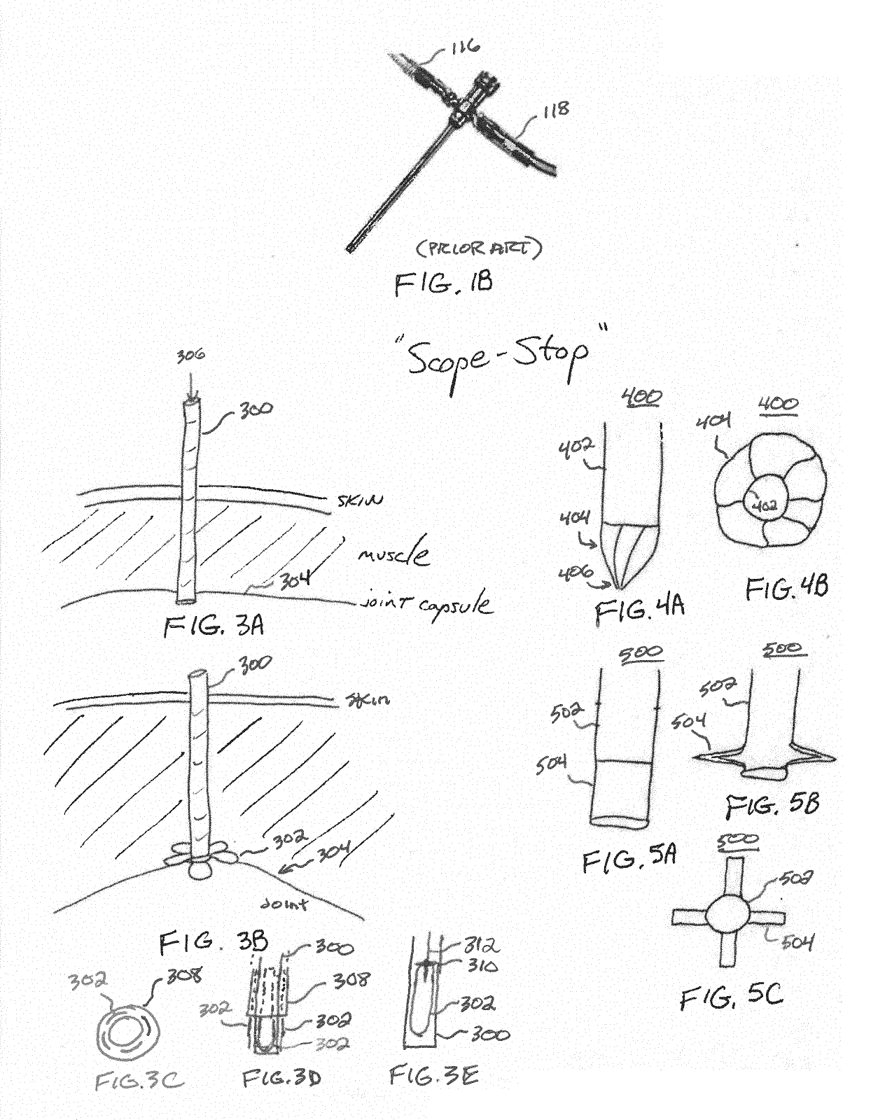

FIGS. 1A and 1B are images of a conventional arthroscopic instrument used to enable a surgeon to view inside of joints, such as a shoulder joint, of a patient;

FIG. 2 is an illustration of an illustrative working cannula that is used for inserting an instrument;

FIG. 3A is an illustration of an arthroscopic cannula that is shown to extend through skin, muscle, and joint capsule;

FIG. 3B is an illustration of the arthroscopic camera cannula shown to include expandable flanges that may be used to limit removal of the arthroscopic camera cannula from within a joint once the expandable flanges are inserted and expanded within the joint, in this case a shoulder joint;

FIGS. 3C-3E are illustrations of alternative embodiments of the arthroscopic camera cannula with a sheath that maintains the expandable flanges (FIGS. 3C and 3D) in a closed position and moveable arm that operates to open and close the expandable flanges (FIG. 3E);

FIGS. 4A and 4B are illustrations of an alternative illustrative arthroscopic camera cannula including expandable flanges at an end of the arthroscopic camera cannula;

FIGS. 5A-5C are illustrations of an illustrative arthroscopic camera cannula including alternative expandable flanges at an end of the arthroscopic camera cannula;

FIG. 6 is an image of an illustrative patient undergoing shoulder surgery sitting on a conventional surgical chair or table;

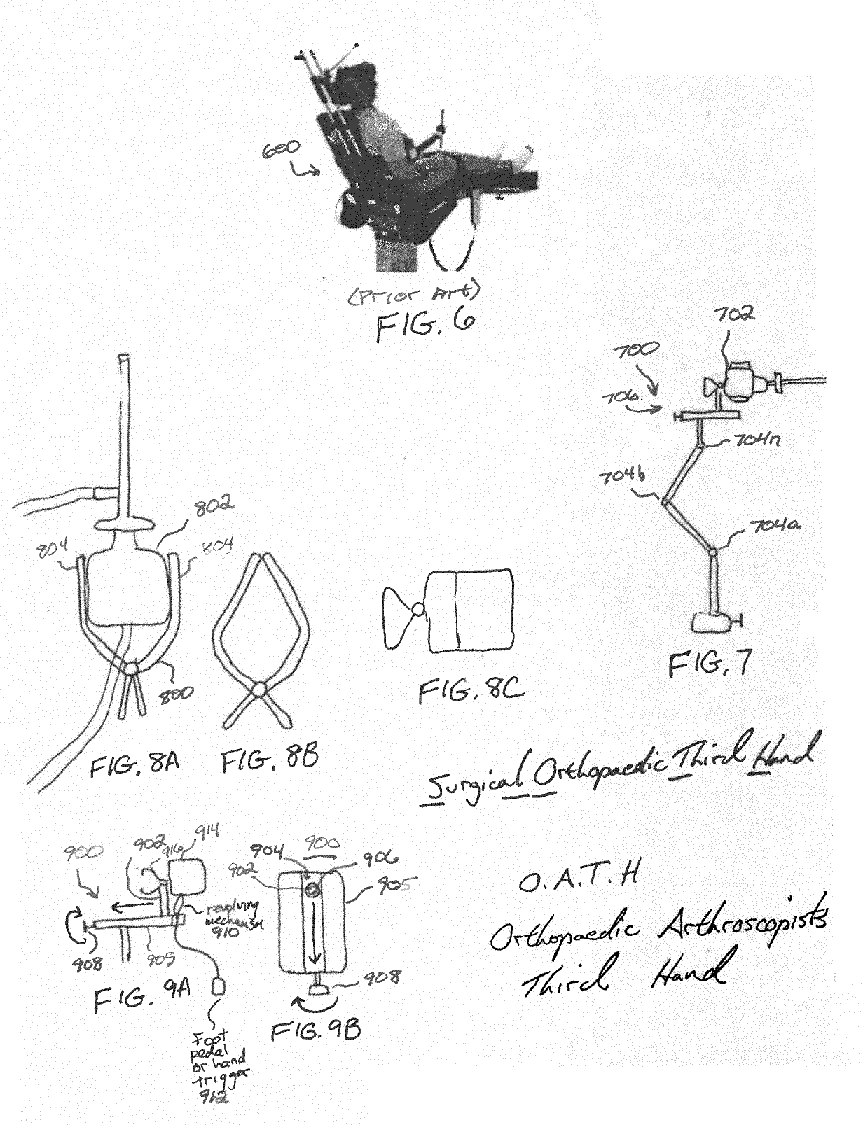

FIG. 7 is an illustration of an articulating bar that may be configured to hold an arthroscopic camera in a fixed position;

FIGS. 8A-8C are illustrations of an illustrative gripper for use on an articulating bar to hold an arthroscopic camera;

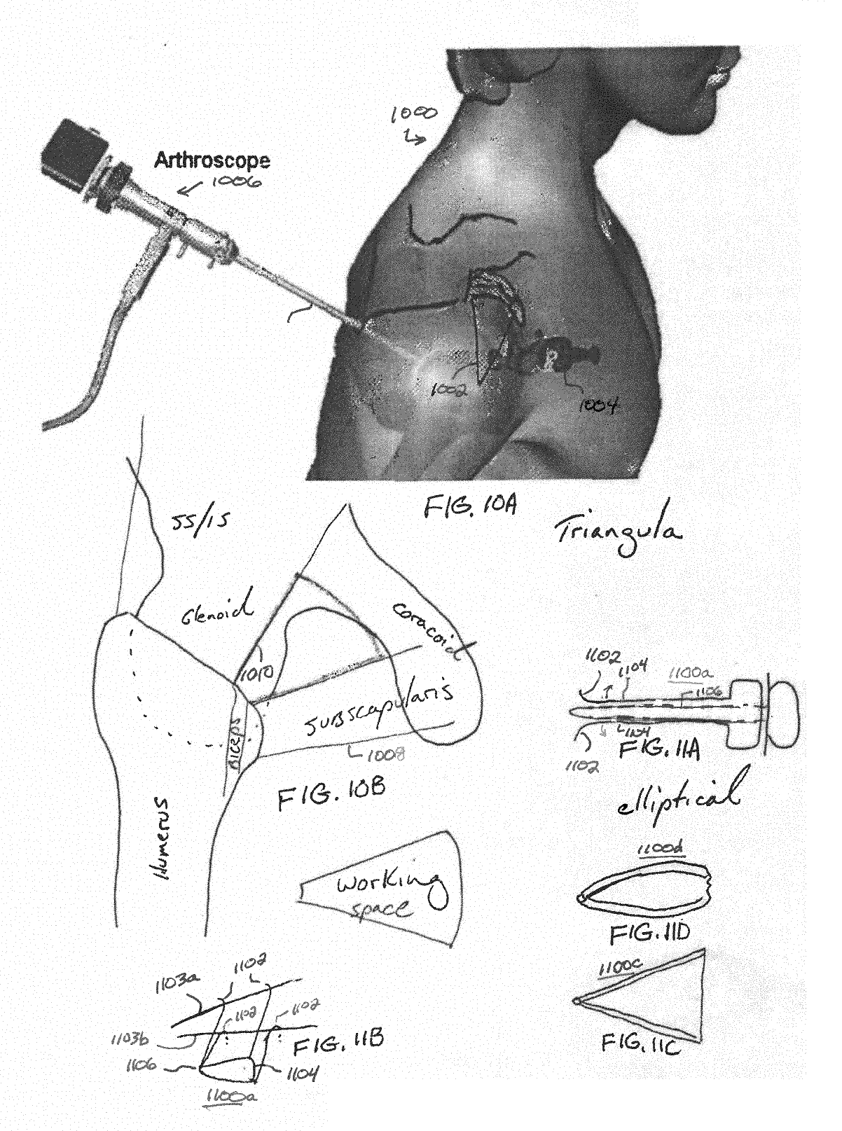

FIGS. 9A and 9B are side and top views of an illustration of a top support member configured to be of the articulating arm of FIG. 7;

FIGS. 10A and 10B are illustrations of an illustrative surgical area in the shape of a triangle or wedge in which arthroscopic shoulder surgery may be performed; and

FIGS. 11A-11C are illustrations of an illustrative arthroscopic working cannula that has a non-circular shape along with “feet” or curved portions at the bottom of adjustable members that extend along the arthroscopic working cannula.

### DETAILED DESCRIPTION OF THE DRAWINGS

With regard to FIGS. 1A and 1B, images of a conventional arthroscopic instrument 100 used to enable a surgeon to view inside of joints, such as a shoulder joint, of a patient is shown. A camera 102 with a cord 104 that is used to collect and communicate image signals to a camera and/or monitor is provided. A scope 106 inclusive of optics is optically coupled to the camera 102 on one side and with an optical tube 108 having a lens 110 at a distal end on another side. A fiber optic light 112 source may be connected to a connector 114 that may include optics, such as a prism, beam splitter reflector, or otherwise, to enable light from the fiber optic light source 112 to be projected along the optical tube 108 so as to illuminate a field-of-view (FOV) of the lens 110 of the arthroscopic instrument. As shown in FIG. 1B, two fluid conduits 116 and 118 are connected to the arthroscopic instrument 100, one to input or inject saline to a joint, and another output or remove fluid and bone fragments from the joint.

As understood in the art, the lens 110 at the end of the optical tube 108 is typically a 30 degree or 70 degree angled lens, although other angular lenses are possible. During an operation, a surgeon typically holds the arthroscopic instrument 100 in one hand and a surgical instrument in another hand, and as he or she is operating, moves the arthroscopic instrument 100 to adjust his or her viewing region. Because of the angled lens 110, the surgeon does not use the arthroscopic instrument to view directly in front of the lens, but rather to an angle (i.e., either 30 degree or 70 degree angle depending on the selected lens).

With regard to FIG. 2, an illustration of an illustrative working cannula 200 that is used for inserting a surgical instrument is shown. In general, the working cannula 200 is not a cannula that is typically used for inserting the optical tube of the arthroscopic device due to being configured for surgical instruments for use during surgery. The working cannula 200 is typically a disposable plastic device that includes an obturator that extends through an opening 202 formed by the working cannula. The working cannula 200 typically has a diameter between 6 mm and 9 mm to be able to accommodate different sized surgical instruments used during surgery. Surgical instruments, such as suture passing devices, shavers, arthroscopic burrs, drill guides, scissors, and so forth are generally passed through the working cannula 200. As understood in the art, an arthroscopic camera, such as the arthroscopic instrument 100 of FIG. 1 is typically not used with a working cannula 200 due to a ratio of the diameter of the working cannula 200 relative to the diameter of the optical tube 108 of the arthroscopic camera 100 being too large. Expandable flanges 204 are included on the working cannula 200 so as to inhibit the ability for the working cannula 200 to be pulled out of tissue, such as skin or muscle, into which the working cannula 200 has been inserted. The flanges 204 of the working cannula 200 enables a surgeon to expand his or her working space by pulling on the working cannula 200, which pulls on the tissue in which the working cannula 200 is extended.

With regard to FIG. 3A, an illustration of an illustrative arthroscopic cannula 300 is shown that extends through skin, muscle, and joint capsule is shown. The arthroscopic cannula 300 is typically 3 ½ mm to 4 mm in diameter, and formed of metal to provide for a rigid guide through which an optical tube of an arthroscopic instrument is inserted. As shown in FIG. 3B, an illustration of the arthroscopic camera cannula 300 is shown to include expandable flanges 302 that may be used to limit removal of the arthroscopic camera cannula from within a joint 304 once inserted and expanded within the joint 304, in this case a shoulder joint. Dimensions and shape of the flanges 302 may be established to provide for usage in a particular joint for different sized patients. To insert the arthroscopic camera cannula 300, an obturator (not shown) that fits down a center opening 306 may puncture an opening into the joint 304 into which the arthroscopic cannula 300 is to be inserted. The obturator is thereafter removed to enable the optical tube of the arthroscopic instrument to be inserted. The flanges 302 may be connected to the arthroscopic cannula 300 on either end.

To expand the expandable flanges 302, a variety of mechanisms may be utilized. In one embodiment, the expandable flanges or wings may have a spring (not shown) that biases the expandable flanges 302 to be in an expanded position. The spring may be positioned within the center opening, outside the cannula 300, or within a wall of the cannula 300. Appropriate connection members may connect to or engaged the spring to bias the flanges 302. In an embodiment, a slidable sheath 308 (FIGS. 3C and 3D) may fit over the metallic, arthroscopic camera cannula 300 and optionally be shorter than the arthroscopic camera cannula 300. The sheath 308 may be formed of plastic or any other material. In an insertion position, the sheath 308 may be positioned over the expandable flanges 302 so as to crimp the expandable flanges 302 against the arthroscopic camera cannula 300. Once inserted into the joint, the sheath 308 may be withdrawn or slid away from the joint 304 to be in a release position, thereby causing the expandable flanges 302 to be released and opened.

In another embodiment, and as shown in FIG. 3E a hinge mechanism 310 connected to the expandable flanges 302 may include a slider member 312 that that operates similar to an umbrella to enable a surgeon to close and open the expandable hinges 302 prior to and after insertion of the arthroscopic camera cannula 300 into a joint. Still yet, a rotational screw member (not shown) may attach to one or more slider members (not shown) that cause the expandable flanges 300 to open and close as the rotational screw member is rotated clockwise and counterclockwise. In yet another embodiment, a ratchet mechanism (not shown) that enables the expandable flanges to open and closed may be utilized. The same or different mechanisms may be utilized to open and close the expandable flanges. A variety of configurations of expandable flange members may be utilized to provide for the functionality of expandable members that resist withdrawal of the arthroscopic camera cannula 300 from within a joint capsule of a patient. By pulling back on the arthroscopic camera cannula 300 against the joint capsule, stability of the arthroscopic camera cannula 300 may be provided along with additional viewing area within the joint capsule 304. Moreover, the number of expandable flange members 302 may vary, such as two or more, and may be diametrically opposed to one another. The number of expandable flange members may depend on the joint, insertion point, specific use, and so on.

With regard to FIGS. 4A and 4B, illustrations of an illustrative arthroscopic camera cannula 400 including an arthroscopic cannula tube 402 with expandable flanges 404 disposed at an end of the arthroscopic camera cannula tube 404 is shown. In a closed position, the expandable flanges form a tip 406, as shown in FIG. 4A. Once the arthroscopic camera cannula tube 402 is entered into a shoulder joint, for example, the expandable flange members 404 may be expanded into an open position. A rotation member, slide member, or other mechanical member (not shown) may be used to expand and collapse the expandable flange members 404 between the closed position and open position.

With regard to FIGS. 5A-5C, illustrations of an illustrative arthroscopic camera cannula 500 including expandable flanges 504 at an end of the arthroscopic camera cannula tube 502 is shown. In a closed position, the expandable flanges 504 extend along a sidewall of the arthroscopic camera cannula tube 502, as shown in FIG. 5A. The expandable flanges 504 may be expanded, as shown in FIG. 5B, where the expandable flanges 504 may be hinged in a center location of the expandable flanges 504. One or more control members (not shown) may cause the expandable flanges 504 to expand and contract radially outward from the arthroscopic camera cannula. FIG. 5C is a bottom view illustration of the arthroscopic camera cannula 500 of FIG. 5B. Size and shape of the expandable flanges 504 may be varied based on a number of factors, but function to provide resistance to withdrawal of the cannula from a joint with a certain force applied. Control mechanisms may include a slidable sheath, ratchet mechanism, rotation mechanism, umbrella mechanism, or other mechanism that causes the expandable flanges 504 to expand (open) and contract (close).

With regard to FIG. 6, an image of an illustrative patient undergoing shoulder surgery is shown to be sitting on a conventional surgical chair or table. As understood in the art, the surgical chair allows for the surgeon to place the patient and arm in any position using a movable arm holder (not shown) as needed for performing a shoulder surgery. The arm holder may be configured to be oriented with 360 degrees of freedom using a foot pedal or hand trigger to control using hydraulics.

With regard to FIG. 7, an illustration of an articulating bar 700 that may be configured to hold an arthroscopic camera 702 in a fixed position is shown. The articulating bar 700 may be configured to be mounted to the chair 600 of FIG. 6, for example. Alternatively, the articulating bar 700 may be configured to be mounted to a floor, mount positioned on the floor, ceiling, or elsewhere that enables a surgeon to position the arthroscopic camera 702 in a desired position to view within a shoulder joint, for example. The articulating arm 700 may have multiple joints 704a-704n, which is three for articulating bar 700. It should be understood, however, that a snake configuration, rather than jointed arm, may alternatively be utilized. It should further be understood that alternative jointed configurations may be utilized. At the top of the articulating arm is a gripper, holder, and/or clamp 706 or overall a support member that is shaped and sized to hold a camera body or other structural feature of the arthroscopic camera 702.

By utilizing the articulating bar 700 to support and position the arthroscopic camera 702 in conjunction with the expandable flanges 302 on the optical cannula 300 (FIG. 3A), for example, a surgeon may be able to place the arthroscopic camera 702 in a desirable position, thereby freeing up one of the hands of the surgeon as the surgeon no longer needs to hold the camera during surgery. Once both hands are freed up, the surgeon may perform arthroscopic surgery in a manner heretofore not possible.

With regard to FIGS. 8A-8C, illustrations of an illustrative gripper 800 for use on an articulating bar, such as articulating bar 700 of FIG. 7, is shown. The gripper 800 may include a pair (or other number) of grip members 804 may expand and squeeze a camera 802 of an arthroscope in a position set by the articulating arm. Alternative configurations of the gripper 800 may be utilized. Alternative configurations of a device that is sized and shaped to hold the camera 802 of the arthroscope may be utilized.

With regard to FIGS. 9A and 9B, side and top views of an illustration of a top support member 900 that may be adapted to be held or supported by the articulating arm 700 of FIG. 7 is shown. The support member 900 may include a post 902 positioned within a track 904 of a support structure 905 having a repositionable member 906 that may travel forward and back with fine adjustment (e.g., at millimeter scales). In an embodiment, a knob 908 that causes a screw drive (not shown) may provide for forward and backward motion of the post 902. The post may be coupled to the arthroscopic camera to cause the camera to move forward and backward so as to expand and reduce field-of-view of a scene within a joint. That is, as a lens at the end of an optical tube of an arthroscopic camera extends out of and into an arthroscopic camera cannula (e.g., arthroscope camera cannula 300 of FIGS. 3A and 3B, even at millimeter distances, field-of-view may be considerably varied. Alternative mechanisms for moving the post 902 forward and backward may be utilized, as well.

To provide further viewing capabilities, an embodiment may provide for rotating the image via the camera, lens, or optical tube. In an embodiment, a foot pedal or hand trigger 912 may be provided to drive the revolving or rotational mechanism 910 if electromechanical or other drive type holding an arthroscopic camera 914 to rotate the viewing angle thereof. In an embodiment, the rotational mechanism may be part of a gripper. Alternatively, the rotational mechanism 916 may extend from the gripper or top support member of the articulating arm. The rotational mechanism 910 may rotate any of the camera, lens, or optical tube to cause the viewing angle to be rotated. The foot pedal 912 may use hydraulics, electromechanical, mechanical, or other drive mechanism to control rotation of the image. In one embodiment, the functionality of rotating the camera or otherwise may be integrated into a foot pedal used for controlling the light source. As an example, the surgeon may press on the foot pedal 912, and the arthroscopic camera may rotate to the left or right, and a 360 degree rotation may be possible over a time period (e.g., 5 to 10 seconds).

When performing conventional shoulder replacement surgery, a surgeon opens skin of the patient so that he or she has full access to the shoulder. As is currently performed during surgery, when the surgeon burrs or reams bone of the shoulder to create a surface onto which a shoulder implant is to be positioned, saline is typically pumped through the arthroscopic cannula into the joint to fill the joint with fluid. Suction via the arthroscopic cannula (outflow hose from arthroscope shown in FIG. 1B) is performed using conventional pressures of wall suction in the operating room to clear blood, bone fragments, and other particulate matter in the joint during the procedure. As understood in the art, wall suction is set at a level that avoids damaging organs, such as liver, kidneys, etc.

In the case of performing arthroscopic surgery, however, higher levels of bone fragments may be created during the arthroscopic burring or arthroscopic reaming process to reshape the undersurface of the scapula. Such a reaming process leads to higher levels of bone fragments being displaced within fluid pumped into the joint, which creates a “snow globe” effect and bleeding. As a result, a surgeon looking at an image created by the arthroscopic camera with the higher levels of bone fragments is slowed down. Hence, in an embodiment, suction pressure via the arthroscopic cannula may be increased above typical wall pressure in the operating room because organs, blood vessels, and other healthy tissue are not of concern due to not being within the surgical area of a shoulder joint. Such higher pressure may be above the blood pressure than the patient's blood pressure, which causes bleeding from the glenoid or humeral head bones to slow down. In an embodiment, the suction pressure may range from a few PSI (e.g., 2 PSI) to many PSI (e.g., 10 PSI or higher), if not higher than the blood pressure of the patient. It should be understood that non-arthroscopic procedures do not have the same problem due to the shoulder being opened, so that bone fragments are able to be displaced outside of the surgical area of the shoulder through open irrigation and suction as well as manual sponge.

During a shoulder operation, after the arthroscopic camera is inserted into a patient's shoulder (see FIG. 10A), the suction pressure may be set to a fixed amount. Typical shoulder arthroscopy inflow via the arthroscopic camera cannula is typically set to about 50 mm of mercury for a pump so that the shoulder is filled with fluid. If no outflow exists from the shoulder, the inflow stops once the pressure in the joint than is at or above 50 mm. The outflow suction keeps the pressure in the shoulder stabilized or the pump is automatically stopped once the desired pressure is reached. In an embodiment, outflow pressure may be adjusted using a valve and/or decrease in pressure level (i.e., higher negative pressure) of a suction pump. In an embodiment, a foot pedal or other control elements (e.g., knob, switch, etc.) available for the surgeon may be used to control input and output pressures to aid the surgeon to clear a field-of-view of blood, bone fragments, etc., within the shoulder during an operation. In an embodiment, a device that includes a timer (e.g., electronic timer, mechanical timer, etc.) may be provided with an activation switch or button to enable a cleaning process for a preset period of time (e.g., 5 seconds).

With regard to FIGS. 10A and 10B, illustrations of a patient 1000 illustrative surgical area 1002 in the shape of a triangle or wedge in which arthroscopic shoulder surgery may be performed are shown. The surgical area 1002 is aligned with the rotator interval. Also shown is a conventional cannula 1004 through which shoulder surgery is typically performed. An arthroscope 1006 is used by the surgeon to see into the shoulder joint during the surgery.

However, in accordance with the principles provided herein, to perform arthroscopic shoulder surgery, the subscapularis tendon 1008 is not cut or detached, as is performed during existing shoulder replacement surgeries. To increase the surgical area between the front rotator cuff tendon (subscapularis) 1008, and the superior rotator cuff tendon (supraspinatus) 1010, the two tendons are to be spread apart. As previously described the conventional working cannula (e.g., working cannula 200 of FIG. 2) has a circular shape as the cannula provide for the limited functionality of insertion of instruments.

With regard to FIGS. 11A and 11B, illustrations of an illustrative arthroscopic working cannula 1100a that has a non-circular shape along with “feet” or curved portions 1102 at the bottom of spreader members 1104 that extend along sidewalls 1106 of the non-circular arthroscopic working cannula 1100a is shown. The feet 1102 may enable the subscapularis tendon 1103a and supraspinatus tendon 1103b to be separated. In an embodiment, the spreader members 1104 may be locked in a spread position using a locking mechanism, such as a pin, latch, or other retention member.

The non-circular shape of the arthroscopic working cannula 1100c may be triangular (see FIG. 11C), although the corners may be rounded, to have a similar shape as the rotator interval (i.e., working space) within which the surgeon has to operate on the shoulder. It should be understood that other non-circular shapes that provide for the surgeon to access the rotator interval may be utilized. For example, a side 1104 opposite the “point” 1106 of the triangle may be rounded to provide further angular access for moving an instrument within the arthroscopic working cannula. Still yet, the shape of an arthroscopic working cannula 1100d may be elliptical, as shown in FIG. 11D.

In operation, the spreader members 1104 that extend along the arthroscopic working cannula 1100a may have “feet” 1102 that are curved to hook onto the two tendons 1103a and 1103b. Once hooked, a spreading mechanism (e.g., ratchet, rotating member, pivot member, or otherwise) may be activated to separate or otherwise spread the two tendons 1003a and 1103b so as to increase the working space for the surgeon. In an embodiment, the spreader members 1104 define sidewalls of the arthroscopic working cannula, thereby reducing the number of components that form the arthroscopic working cannula. It should be understood that other configurations of the orthoscopic working cannula 1100a may be utilized and provide for the functionally described herein.

The arthroscopic working cannula may be formed of plastic or any other material, as understood in the art. The spreading members may be plastic or other material. A variety of configurations that provide for the spreading members to be spread and close may be utilized. As an example, a hinge, screw drive, plastic deformable connection with a lock, jack, or other mechanism that allows for spreading the spreading members and locking the members in place once spread may be utilized. And, because the arthroscopic working cannula may be triangular in shape, the spreading members may also be triangular in shape to be similarly shaped to the rotator interval, thereby reducing complexity or eliminating a more complex instrument that would otherwise be used in addition to a conventional working cannula.

As understood in the art, a largest diameter head of instruments used for performing shoulder surgery are about 53 mm. Hence, longitudinal and latitudinal distances within the arthroscopic working cannula 1100a, 1100c, or 1100d are to be large enough to enable instruments, and shoulder implantable reconstruction members, to be inserted therethrough.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the scope of the invention. Thus, the present invention is not intended to be limited to the embodiments shown herein, but is to be accorded the widest scope consistent with the following claims and the principles and novel features disclosed herein.

What is claimed is:

1. An arthroscopic camera cannula, comprising:
a hollow, elongated member having a first end and a second end, the elongated member having a diameter less then approximately 4 mm to enable an arthroscopic camera optical tube to be inserted therethrough;
a plurality of expandable flanges being coupled to the hollow, elongated member toward the second end; and
a mechanism in mechanical communication with said expandable flanges that, when moved, causes said expandable flanges to move between a closed position and an open position.

2. The arthroscopic camera cannula according to claim 1, wherein the mechanism is configured to rotate.

3. The arthroscopic camera cannula according to claim 1, wherein the mechanism slides along the hollow, elongated member.

4. The arthroscopic camera cannula according to claim 1, wherein said flanges form a tip in a closed position.

5. The arthroscopic camera cannula according to claim 1, wherein said flanges transition from a closed position to an open position simultaneously.

6. The arthroscopic camera cannula according to claim 1, further comprising a sheath that is configured to extend over said hollow, elongated member and said expandable flanges.

7. An arthroscopic camera support stand, comprising:
an articulating bar including a plurality of joints between a base end and a retention end;
a retention member attached to the retention end of said articulating bar, and configured to retain an arthroscopic camera, the retention member including:
a first mechanism configured to retain the arthroscopic camera; and
a second mechanism configured to longitudinally adjust position of the arthroscopic camera.

8. The support stand according to claim 6, further comprising a rotational member attached to said retention member, and configured to rotate the arthroscopic camera.

9. The support stand according to claim 7, wherein the rotational member is electromechanical.

10. An arthroscopic instrument, comprising:
a housing having an inlet port and an outlet port;
a first tube connected to the inlet port for inserting fluid into the inlet port to be fluidly transported into a joint of a patient;
a second tube connected to the outlet port, and configured to apply a vacuum pressure to the outlet port; and
a vacuum applied to the second tube to apply a vacuum pressure below a vacuum pressure available from a wall vacuum in an operating room.

11. An arthroscopic working cannula, comprising:
a cannula member;
a first spreader member attached to and extended radially along said cannula member, said first spreader member having a first end and a second end, the second end having a curved shape to hook onto the subscapularis tendon; and
a second spreader member attached to and extended radially along said cannula member, said second spreader member having a first end and a second end, the second end having a curved shape to hook onto the supraspinatus tendon, the first and second spreader members being disposed in opposing position relative to one another, and, when spread from a closed position to an open position, form an expanded triangular area between the subscapularis and supraspinatus tendons.

12. The arthroscopic working cannula according to claim 11, wherein said first and second spreader members define sidewalls of the cannula, and wherein the cannula member defines a top portion of the cannula.

13. The arthroscopic working cannula according to claim 11, wherein said first and second spreader members extend along sidewalls of said cannula member.

14. The arthroscopic working cannula according to claim 11, wherein said first cannula member has a pair of sidewalls having an angle greater than 10 degrees between the sidewalls.

15. The arthroscopic working cannula according to claim 11, wherein said first and second spreader members have an angle of greater than 10 degrees when in the closed position.
